(19) United States
(12) Patent Application Publication
(10) Pub. No.: US 2022/0079197 A1
(43) Pub. Date: Mar. 17, 2022

(54) FLAVOUR MODIFYING INGREDIENT DERIVED FROM DIETARY FIBRE

(71) Applicant: Givaudan SA, VERNIER (CH)

(72) Inventors: Tarun BHOWMIK, Mason, OH (US); Stephanie Ann Sander LINCH, Lebanon, OH (US); Stefka Ivanova MYAKA, Cincinnati, OH (US)

(21) Appl. No.: 17/423,315

(22) PCT Filed: Jan. 30, 2020
(86) PCT No.: PCT/EP2020/052312

§ 371 Date: Jul. 15, 2021

Related U.S. Application Data
(60) Provisional application No. 62/800,413, filed on Feb. 1, 2019.

(51) Int. Cl. A23L 27/00; C12N 1/20; A23L 7/104; C12N 9/48; C12N 9/78; A23L 33/21; A23C 9/123; A23C 11/10

(52) U.S. Cl. CPC A23L 27/88; C12N 1/20; A23L 7/104; C12N 9/48; C12R 2001/25; A23L 33/21; A23C 9/1234; A23C 11/106; C12N 9/78

(57) ABSTRACT

A method for making a flavour modifying ingredient, the method comprising subjecting a dietary fibre to enzymatic hydrolysis and/or fermentation; flavour modifying ingredients obtainable by said method; flavour compositions and food products comprises said flavour modifying ingredient; uses of said flavour modifying ingredient.

### TECHNICAL FIELD

The present invention relates generally to methods for making flavour modifying ingredients using dietary fibre and the flavour modifying ingredients made by said methods. The present invention further relates to flavour compositions and food compositions comprising said flavour modifying ingredients and the uses of said flavour modifying ingredients in food compositions, for example to improve mouthfeel of the food composition and/or mask off-notes of the food composition and/or improve sweetness of the food composition and/or enhance saltiness of the food composition.

### BACKGROUND

There exists a need in the food industry to provide ingredients which can modify the flavour of various food products, for example to improve the mouthfeel, mask off-notes, improve sweetness, and/or enhance saltiness. In particular, a need exists to provide flavour modifying ingredients which are natural and/or suitable for vegans. Novel flavour modifying ingredients and methods for making said flavour modifying ingredients are therefore provided by the present invention.

### SUMMARY OF THE INVENTION

In accordance with a first aspect of the present invention there is provided a method for making a flavour modifying ingredient, the method comprising subjecting a dietary fibre to enzymatic hydrolysis and/or fermentation.

The method described in WO 2010/053653 A1 and the method described in US 2009/0311376 A1 are excluded from the method of the first aspect of the present invention.

For example, the method of the first aspect of the present invention may exclude a method comprising (a) contacting a fiber-digesting enzyme with a suspension comprising an amount of water and cleaned whole grain oat flour, and (b) treating the suspension for a period of time sufficient to hydrolyze fiber particles such that a modified whole grain oat flour is formed. For example, the method of the first aspect of the present invention may exclude a method comprising contacting a fiber-digesting enzyme with a suspension comprising an amount of water and cleaned whole grain oat flour.

For example, the method of the first aspect of the present invention may exclude a method comprising combining a whole oat or barley flour starting mixture and a suitable enzyme to form an enzyme starting mixture, heating the enzyme starting mixture to between about 120° F. and about 200° F. to begin to hydrolyze the starch molecules, and extruding the resultant mixture to continue hydrolyzing the starch and further to gelatinize and cook the mixture to form the soluble oat or barley flour. For example, the method of the first aspect of the present invention may exclude a method comprising combining a whole oat or barley flour starting mixture and a suitable enzyme to form an enzyme starting mixture, and heating the enzyme starting mixture to between about 120° F. and about 200° F. to begin to hydrolyze the starch molecules.

For example, the dietary fibre may not be cleaned whole grain oat flour. For example, the dietary fibre may not be whole oat flour and/or may not be oat flour and/or may not be barley flour. For example, the dietary fibre may not be oat flour and/or may not be barley flour. For example, the dietary fibre may not be oat fibre and/or may not be barley fibre.

In certain embodiments, the dietary fibre is isolated dietary fibre. In certain embodiments, the dietary fibre is an aqueous slurry of dietary fibre.

In certain embodiments, the dietary fibre is a cereal fibre (e.g. oat fibre), a vegetable fibre (e.g. pea fibre), or a fruit fibre (e.g. citrus fruit fibre, apple fibre, blueberry fibre, cranberry fibre, grape fibre).

In certain embodiments, the enzymatic hydrolysis uses one or more enzymes selected from carbohydrases and proteolytic enzymes. In certain embodiments, the enzymatic hydrolysis uses at least one or more enzymes selected from cellulases, pectinases, and other carbohydrases.

In certain embodiments, the fermentation uses a lactic acid bacterium (e.g. Lactobacillus plantarum, L. delbrueckii ssp. bulgaricus, Streptococcus thermophiles and/or Lactobacillus acidophilus) and/or a Bifidobacterium and/or an Aspergillus fungus (e.g. Aspergillus oryzae).

In certain embodiments, the enzymatic hydrolysis is performed at a temperature ranging from about 25° C. to about 60° C.

In certain embodiments, the enzymatic hydrolysis takes place for a period of time ranging from about 1 hour to about 48 hours.

In certain embodiments, the fermentation is performed at a temperature ranging from about 20° C. to about 45° C.

In certain embodiments, the fermentation takes place for a period of time ranging from about 1 day to about 10 days.

In certain embodiments, the method of the first aspect of the present invention comprises subjecting the dietary fibre to enzymatic hydrolysis and fermentation. In certain embodiments, the enzymatic hydrolysis takes place before and/or simultaneously with the fermentation.

In certain embodiments, the method of the first aspect of the present invention comprises subjecting a dietary fibre to enzymatic hydrolysis and does not comprise subjecting the dietary fibre to fermentation.

In certain embodiments, the method of the first aspect of the present invention comprises subjecting a dietary fibre to fermentation and does not comprise subjecting the dietary fibre to enzymatic hydrolysis.

In certain embodiments, the method of the first aspect of the present invention further comprises heating the dietary fibre to a temperature equal to or greater than about 75° C. prior to the enzymatic hydrolysis and fermentation.

In certain embodiments, the method of the first aspect of the present invention further comprises deactivating the enzyme and/or the fermentation microorganism following the enzymatic hydrolysis and/or fermentation.

In certain embodiments, the method of the first aspect of the present invention further comprises combining the flavour modifying ingredient with propylene glycol.

In certain embodiments, the method of the first aspect of the present invention further comprises spray-drying the flavour modifying ingredient.

In accordance with a second aspect of the present invention there is provided a flavour modifying ingredient obtainable by and/or obtained by the method of the first aspect of the present invention, including any embodiment therefore.

In accordance with a third aspect of the present invention there is provided a flavour composition comprising the flavour modifying ingredient of the second aspect of the present invention.

In accordance with a fourth aspect of the present invention there is provided a food product comprising the flavour modifying ingredient of the second aspect of the present invention.

In accordance with the fifth aspect of the present invention there is provided the use of a flavour modifying ingredient of the second aspect of the present invention to improve the mouthfeel of a food product.

In accordance with a sixth aspect of the present invention there is provided a method of providing a food product having an improved mouthfeel, the method comprising admixing the flavour modifying ingredient of the second aspect of the present invention to the food product.

In accordance with the seventh aspect of the present invention there is provided the use of a flavour modifying ingredient of the second aspect of the present invention to mask off-notes of a food product.

In accordance with a eighth aspect of the present invention there is provided a method of providing a food product having reduced off-notes, the method comprising admixing the flavour modifying ingredient of the second aspect of the present invention to the food product.

In accordance with the ninth aspect of the present invention there is provided the use of a flavour modifying ingredient of the second aspect of the present invention to improve the sweetness of a food product.

In accordance with a tenth aspect of the present invention there is provided a method of providing a food product having improved sweetness, the method comprising admixing the flavour modifying ingredient of the second aspect of the present invention to the food product.

In accordance with the eleventh aspect of the present invention there is provided the use of a flavour modifying ingredient of the second aspect of the present invention to enhance the saltiness of a food product.

In accordance with a twelfth aspect of the present invention there is provided a method of providing a food product having enhanced saltiness, the method comprising admixing the flavour modifying ingredient of the second aspect of the present invention to the food product.

In certain embodiments of any aspect of the present invention, the food product is a dairy product or a dairy alternative product or a beverage or a savoury food.

In certain embodiments of any aspect of the present invention, the food product further comprises one or more sweeteners. In certain embodiments, the one or more sweeteners are selected from sucrose, fructose, glucose, arabinose, rhamnose, tagatose, allulose, trehalose, isomaltulose, steviol glycosides (e.g. rebaudioside A, rebaudioside B, rebaudioside C, rebaudioside D, rebaudioside M, stevioside), stevia, trilobatin, rebusoside, aspartame, advantame, agarve syrup, acesulfame potassium (AceK), high fructose corn syrup, neotame, saccharin, sucralose, high fructose corn syrup, starch syrup, Luo Han Guo extract, mogrosides, neohespiridin, dihydrochalcone, naringin, and sugar alcohols (e.g. sorbitol, xylitol, inositol, mannitol, erythritol).

Certain embodiments of any aspect of the present invention may provide one or more of the following advantages:
production of a natural product;
food product with improved mouthfeel;
food product with reduced off-notes;
food product with improved sweetness;
food product with enhanced saltiness;
dairy alternative product with improved dairy-like characteristics.

The details, examples and preferences provided in relation to any particulate one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based, at least in part, on the surprising finding that subjecting dietary fibre to enzymatic hydrolysis and/or fermentation produces a product that can be used as a flavour modifying ingredient, for example to improve the mouthfeel of a food product, to mask off-notes of a food product, to improve the sweetness of a food product, and/or to enhance the saltiness of a food product.

In particular, the present invention is based, at least in part, on the surprising finding that the flavour modifying ingredients described herein can be used to eliminate the unpleasant beany taste of dairy alternative products, provide a “fullness” sensation to low-fat or non-fat dairy products that is similar to the corresponding full-fat dairy product, and can enhance saltiness in savoury food products such as chips. Dietary fibre has previously been used in food products for a bulking effect so it is surprising that the flavour modifying ingredients described herein provide the advantageous taste and mouthfeel effects described herein.

In certain embodiments, the dietary fibre is subjected to enzymatic hydrolysis and not to fermentation. Where the dietary fibre is subjected to enzymatic hydrolysis and not to fermentation, the dietary fibre may, for example be a fruit fibre such as grape fibre. In certain embodiments, the dietary fibre is subjected to fermentation and not to enzymatic hydrolysis. Where the dietary fibre is subjected to fermentation and not to enzymatic hydrolysis, the dietary fibre may, for example, be a cereal fibre such as an oat fibre. In certain embodiments, the dietary fibre is subjected to enzymatic hydrolysis and fermentation, for example wherein enzymatic hydrolysis occurs before and/or simultaneously with fermentation.

### Dietary Fibre

The term “dietary fibre” refers to a type of carbohydrate that cannot be completely broken down by human digestive enzymes. It is found in edible plant foods such as cereals, fruits, vegetables, nuts, seeds, lentils, fungi, and grains.

The term “dietary fibre” includes non-starch polysaccharides, resistant starch, cellulse, hemicellulose, psyllium, dextrins, inulin, lignins, lichenin, chitins, pectins, beta-glucans, and oligosaccharides. The dietary fibre may, for example, be soluble fibre or insoluble fibre.

The dietary fibre may, for example, be a cereal fibre, a vegetable fibre, a fruit fibre, a nut fibre, a seed fibre, a lentil fibre, a fungi fibre, or a grain fibre. The dietary fibre may, for example, be a cereal fibre, a vegetable fibre, or a fruit fibre. The terms “cereal fibre”, “vegetable fibre”, and “fruit fibre” refer to types of fibres that are obtained and/or obtainable respectively from a cereal, a vegetable, or a fruit.

The dietary fibre may, for example, be obtained and/or obtainable from one or more types of plant. The dietary fibre may, for example, be obtained and/or obtainable from fresh, dried, or rehydrated plant material.

The dietary fibre may, for example, be isolated dietary fibre. The term “isolated dietary fibre” refers to dietary fibre that is separate to the plant in which it is found.

The dietary fibre may, for example, be a side-stream from an industrial process, for example a side-stream from juice production. This may, for example, provide environmental advantages.

Cereal fibre includes, for example, oat fibre, maize fibre, rice fibre, wild rice fibre, wheat fibre, barley fibre, soghum fibre, millet fibre, rye fibre, triticale fibre, and fonio fibre. In certain embodiments, the cereal fibre is oat fibre. The fibre may, for example, be obtained and/or obtainable from the seed of the plant.

Vegetable fibres include, for example, legume fibre such as pea fibre, chickpea fibre, lentil fibre, and soybean fibre; root vegetable fibre such as potato fibre, sweet potato fibre, carrot fibre, celeriac fibre, parsnip fibre, radish fibre, and onion fibre; broccoli fibre; cabbage fibre; green bean fibre; cauliflower fibre; courgette fibre; and celery fibre. In certain embodiments, the vegetable fibre is legume fibre such as pea fibre. The fibre may, for example, be obtained and/or obtainable from the flower, fruit, stem, leaves, roots, and/or seeds of the plant.

Fruit fibres include, for example, citrus fruit fibre such as orange fibre, lemon fibre, lime fibre, clementine fibre, tangerine fibre, grapefruit fibre, kumquat fibre, yuzu fibre; apple fibre; grape fibre; tomato fibre; bell pepper fibre; cucumber fibre; berry fibre such as blueberry fibre, cranberry fibre, strawberry fibre, raspberry fibre, blackberry fibre, red currant fibre, white current fibre, and blackcurrent fibre; avocado fibre; fig fibre; plum fibre; prune fibre; banana fibre; pear fibre; and kiwi fibre. In certain embodiments, the fruit fibre is cranberry fibre, grape fibre, or a combination of one of more thereof. The fibre may, for example, be obtained from the fruit of the plant.

Where the dietary fibre is subjected to enzymatic hydrolysis but not fermentation, the dietary fibre may be a fruit fibre such as citrus fruit fibre, apple fibre, blueberry fibre, cranberry fibre, grape fibre.

Where the dietary fibre is subjected to fermentation but not enzymatic hydrolysis, the dietary fibre may be a cereal fibre such as oat fibre.

### Enzymatic Hydrolysis

In certain embodiments, dietary fibre is subjected to enzymatic hydrolysis, wherein the dietary fibre is contacted with one or more enzyme(s) under conditions and for a period of time suitable for the enzyme(s) to at least partially break down the dietary fibre. All enzymes should be food grade.

The enzyme(s) used for enzymatic hydrolysis may, for example, be selected from one or more of carbohydrases and proteolytic enzymes. Where more than one enzyme is used, the enzymes may be more than one class of enzymes and/or more than one enzymes within a single class. In certain embodiments, the enzyme(s) used for enzymatic hydrolysis include at least one or more carbohydrase(s). In certain embodiments, the enzyme(s) used for enzymatic hydrolysis include at least one or more of cellulases, pectinases, and other carbohydrases. In certain embodiments, the enzyme(s) used for enzymatic hydrolysis include at least one or more of cellulases and pectinases.

Carbohydrases catalyse the hydrolysis of carbohydrates. The carbohydrases may have specificity for either alpha- or beta-glycosidic bonds. Carbohydrases include, for example, cellulases, pectinases, mannanase, amylase, lactase, and beta-glucanase. Examples of amylase enzymes include but are not limited to (i) alpha-amylase enzyme (Kleistase® SD-80, from Amano Enzyme) which is useful in to break down amylose and amylopectin to maltose and various dextrins and/or (ii) Glucoamylase (Gluczyme®NLP from Amano Enzyme) which is useful for example, for the breakdown of maltose and various to release glucose.

Cellulases catalyse the hydrolysis of beta-1,4-glycosidic bonds found in cellulose, hemicellulose, lichenin, and cereal beta-glucans. Cellulases include, for example, hemicellulase, endo-1,4-beta-D-glucanase, xylanase, and carboxymethyl cellulase.

Pectinases catalyse the hydrolysis of alpha-1,4-glycosidic bonds between galacturonic acid residues found in pectin. An example of a pectinase is polygalacturonase (EC 3.2.1.15).

Proteolytic enzymes catalyse the hydrolysis of proteins and peptides. Proteolytic enzymes include, for example, proteinases, which hydrolyze proteins to form small peptides, and peptidases, which further hydrolyze small peptides to form amino acids. The proteolytic enzyme(s) may, for example, have endopeptidase activity (attack internal peptide bonds) and/or exopeptidase activity (attack peptide bonds at the end of the protein or peptide such as amino- or carboxypeptidases).

Proteolytic enzymes include, for example, protease, peptidase, glutaminase (e.g. L-glutamine-amido-hydrolase (EC 3.5.1.2)), endoprotease, serine endopeptidase, subtilisin peptidase (EC 3.4.21.62), serine protease, threonine protease, cysteine protease, aspartic acid protease, glutamic acid protease, trypsin, chymotrypsin (EC 3.4.21.1), pepsin, papain, and elastase.

Proteolytic enzymes (EC 3.4 and EC 3.5) are classified by an EC number (enzyme commission number), each class comprises various known enzymes of a certain reaction type. EC 3.4 comprises enzymes acting on peptide bonds (peptidases/proteinases) and EC 3.5 comprises enzymes that act on carbon-nitrogen bonds other than peptide bonds.

Examples for EC 3.4 include, for example, the following: aminopeptidase (EC 3.4.11), dipeptidase (3.4.13), dipeptidyl-peptidase (3.4.14), peptidyl-dipeptidase (3.4.15), serine-carboxypeptidase (3.4.16), metallocarboxypeptidase (3.4.17), cysteine-carboxypeptidase (3.4.18), omegapeptidase (3.4.19), serine-endopeptidase (3.4.21), cysteine-endopeptidase (3.4.22), aspartate-endopeptidase (3.4.23), metalloendopeptidase (3.4.24), threonine-endopeptidase (3.4.25).

Examples for EC 3.5 include, without limitation, proteolytic enzymes that cleave in linear amides (3.5.1), for example, without limitation, glutaminase (EC 3.5.1.2) and protein glutaminase (eg protein Glutaminase®500 from Amano)

Various proteolytic enzymes, suitable for food-grade applications, are commercially available from suppliers such as Novozymes, Amano, Biocatalysts, Bio-Cat, Valey Research (now subsidiary of DSM), EDC (Enzyme Development Corporation), and others. Some examples include: Neutrase®, Alcalase®, Protamex®, and Flavorzyme®, (available from Novozymes); the Promod® series: e.g. 215P, 278P, 279P, 280P, 192P, and 144P, Flavorpro® 192, Peptidase 433P, and Peptidase 436P (available from Biocatalysts); Protin PC10, Umamizyme®, Peptidase R (or 723), Peptidase A, Peptidase M, Peptidase N, Peptidase P, Peptidase S, Acid protease II, and Thermoase GL30 (available from Amano); Peptidase 600 (available from Bio-Cat); Validase® AFP and Validase® FPII (available from Valey Research); Fungal protease, Exo-protease, Papain, Bromelain, and the Enzeco® series of proteases and peptidases (available from EDC).

In certain embodiments, the enzymes used for enzymatic hydrolysis comprise cellulase, beta-glucanase, and aminopeptidase. In certain embodiments, the enzymes used for enzymatic hydrolysis comprise cellulase, beta-glucanase, aminopeptidase, hemicellulose, and mannanase. In certain embodiments, the enzymes used for enzymatic hydrolysis comprise carbohydrases (such as alpha-amylase and/or glucoamylase) and proteases and/or aminopeptidases (such as protein glutaminase).

The enzymes may be part of an enzyme mix. A number of enzyme preparations such as Celluclast™, Ceramix™, Alcalase™, Viscozyme™, Flavorzyme™, and Umamizyme™, are commercially available and may be used in the enzymatic hydrolysis described herein.

The enzyme(s) may, for example, be obtained or obtainable from a microbial or plant source. Examples include Aspergillus oryzae, Bacillus licheniformis, pineapple, and papaya.

The amount of enzyme is chosen to ensure sufficient activity and depends on the activity of the enzyme, amount of substrate, and conditions it is used in. The necessary amount of enzyme can be determined by trying out different amounts and testing the effect of the resulting product in a sensory evaluation as described herein.

The ratio of enzyme: substrate may, for example, range from about 0.05:20 to about 3:20, for example from about 0.5:20 to about 3:20, for example around 1:20. The enzymes may, for example, be used in an amount ranging from about 0.1 wt % to about 20 wt % based on the total weight of the dietary fibre. For example, the enzymes may be used in an amount ranging from about 0.5 wt % to about 15 wt % or from about 1 wt % to about 10 wt % or from about 0.5 wt % to about 5 wt % or from about 0.5 wt % to about 1.5 wt % or from about 1 wt % to about 1.5 wt % based on the total weight of the dietary fibre.

(Ceremix™, Novozymes, Bagsvaerd, Denmark, has an activity of 300 Beta-Glucanase Units (BGU) per gram of enzyme; Viscozyme™, Novozymes, Bagsvaerd, Denmark, has an activity of 100 Fungal Beta-Glucanase Units FBG per gram of enzyme; Alcalase™, Novozymes, Bagsvaerd, Denmark, has an activity of 2.4 Anson units (AU) per gram of enzyme; Celluclast™, Novozymes, Bagsvaerd, Denmark, has an activity of 700 Endo-Glucanase Units (EGU) per gram of enzyme; Flavourzyme™, Novozymes, Bagsvaerd, Denmark, has an activity of 1000 Leucine Aminopeptidase Units (LAPU) per gram of enzyme; Umamizyme™, Amano, Nagoya, Japan, has an activity of 70 U (Units by LGG method, LGG=L-Leucyl-Glycyl-Glycine); Flavorpro 373™, a Glutaminase, Biocatalysts, Cardiff, UK, has an activity of 30 Glutaminase Units (GU)).

Useful amounts of enzyme units per gram starting material are indicated for some type of enzymes below.

Beta-Glucanase Units (BGU) per gram starting material (liquified celery slurry) 0.03 to BGU, for example 0.1 to 3 BGU.

Fungal Beta-Glucanase Units FBG per gram starting material, 0.002 to 3 FBG, for example, 0.01 to 1 FBG.

Anson units (AU) per gram starting material, 0.0002 to 0.02 AU, for example 0.0005 to 0.01.

U (Units by LGG method, LGG=L-Leucyl-Glycyl-Glycine) per gram starting material 0.007 to 0.7 U, for example, 0.01 to 0.1 U are used.

Glutaminase Units (GU) per gram starting material, 0.00075 to 0.075 GU, for example, 0.001 to 0.02 GU are used.

The enzymatic hydrolysis will be performed under conditions suitable for all the enzymes involved (and all microorganisms involved if occurring simultaneously with fermentation). As will be apparent to the skilled person, the temperature and pH should be within a suitable range for hydrolysis to occur to the desired degree. The incubation length will vary accordingly, with shorter incubations when conditions are nearer to the optimum conditions. Necessary ions, if required or beneficial for the chosen enzymes may be present. Subjecting the incubated mixture to agitation, for example by stirring (e.g. at 50 to 500 rpm or 100 to 200 rpm) may improve the hydrolysis.

The enzymatic hydrolysis may, for example, be performed at a temperature less than the temperature at which the enzymes denature. The temperature may, for example, be selected to give a desired reaction rate. The enzymatic hydrolysis may, for example, be performed at a temperature ranging from about 25° C. to about 60° C. For example, the enzymatic hydrolysis may be performed at a temperature ranging from about 30° C. to about 60° C. or from about 35° C. to about 55° C. or from about 40° C. to about 50° C. or from about 50° C. to about 55° C.

Where the dietary fibre is subjected to enzymatic hydrolysis but not fermentation, the enzymatic hydrolysis may, for example, be performed at a temperature ranging from about 40° C. to about 60° C.

Where the dietary fibre is subjected to enzymatic hydrolysis and fermentation, the enzymatic hydrolysis may, for example, be performed at a temperature ranging from about 30° C. to about 60° C., for example from about 30° C. to about 40° C. or from about 50° C. to about 55° C.

The enzymatic hydrolysis may, for example, be performed at a pH at which the enzymes do not denature. The pH may, for example, be selected to give a desired reaction rate. The enzymatic hydrolysis may, for example, be performed at a pH ranging from about 4 to about 8, for example from about 5 to about 8, for example from about 6 to about 8, for example from about 6.5 to about 7.5.

The enzymatic hydrolysis may, for example, take place for a period of time ranging from about 1 hour to about 48 hours. For example, the enzymatic hydrolysis may take place for a period of time ranging from about 2 hours to about 48 hours or from about 4 hours to about 36 hours or from about 6 hours to about 24 hours or from about 8 hours to about 16 hours or from about 1-2 hours or up to 5 hours.

Where the dietary fibre is subjected to enzymatic hydrolysis and not fermentation, the enzymatic hydrolysis may take place for a longer period of time compared to a method in which the dietary fibre is subjected to enzymatic hydrolysis and fermentation. For example, where the dietary fibre is subjected to enzymatic hydrolysis and not fermentation, the enzymatic hydrolysis may take place for a period of time that is at least about 12 hours, for example at least about 18 hours or at least about 24 hours. For example, where the dietary fibre is subjected to enzymatic hydrolysis and not fermentation, the enzymatic hydrolysis may take place for a period of time ranging from about 12 hours to about 48 hours or from about 18 hours to about 48 hours or from about 24 hours to about 48 hours.

Where the dietary fibre is subjected to enzymatic hydrolysis and fermentation, the enzymatic hydrolysis may take place for a shorter period of time compared to a method in which the dietary fibre is subjected to enzymatic hydrolysis only. For example, where the dietary fibre is subjected to enzymatic hydrolysis and fermentation, the enzymatic hydrolysis may take place for a period of time ranging from about 1 hour to about 36 hours or from about 2 hours to about 36 hours or from about 4 hours to about 24 hours or from about 1-2 hours or up to 5 hours.

### Fermentation

In certain embodiments, dietary fibre is subjected to fermentation, wherein the dietary fibre is contacted with one or more fermenting microorganism(s) under conditions and for a period of time suitable for the microorganism(s) to at least partially break down/metabolize the dietary fibre. Where the dietary fibre was subjected to enzymatic hydrolysis prior to fermentation, the dietary fibre is the product of the enzymatic hydrolysis (a dietary fibre hydrolyzate). The dietary fibre that is the product of the enzymatic hydrolysis may be referred to as hydrolyzed or partly hydrolyzed dietary fibre.

The fermentation may, for example, use one or more species of microorganism.

The fermentation may, for example, use one or more lactic acid bacteria such as Lactobacillus plantarum, Lactobacillus casei, Lactobacillus brevis, and Lactobacillus helveticus. In certain embodiments, the fermentation uses Lactobacillus plantarum. For example, the fermentation may use Lactobacillus plantarum, ATCC 14917.

The fermentation may, for example, use one or more lactic acid bacteria such as L. delbrueckii ssp. bulgaricus, Streptococcus thermophiles and/or Lactobacillus acidophilus. The fermentation may, for example, use a Bifidobacterium.

The fermentation may, for example use an Aspergillus fungus such as Aspergillus oryzae (also known as Koji) and Aspergillus saitoi. In certain embodiments, the Aspergillus fungus is Aspergillus oryzae.

The fermentation may use an overnight culture of the microorganism(s), or the dietary fibre (or dietary fibre hydrolysate obtained from the enzymatic hydrolysis step) may be directly inoculated with a microorganism clone, and the fermentation performed for a slightly longer time accordingly.

The overnight culture (sometimes referred to as seed ferment) may be prepared by methods well-known in the art. It may be grown overnight, for example 12 hours, at the appropriate temperature for that microorganism. Approximately 37° C. is an appropriate temperature for many microorganisms, including Lactobacillus plantarum, L. delbrueckii ssp. bulgaricus, Streptococcus thermophilus, Lactobacillus acidophilus and/or a Bifidobacterium and/or Aspergillus oryzae. Any suitable medium may be used, for example, MRS broth (Difco, United States of America).

The microorgansism(s) may, for example, be administered on a carrier. For example, the microorganism(s) (e.g. Aspergillus oryzae) may be coated onto rice grains. For example, the microorganism(s) may be grown on rice grains and offered by suppliers in this form (e.g. obtainable from Rhapsody Natural Foods, Cabot VT 05647). This may, for example, induce the production of certain endogenous enzymes and/or pathways, thereby providing the microorganism(s) with desirable characteristics.

The amount of microorganism is chosen to ensure sufficient activity and depends on the activity of the microorganism, amount of substrate, and conditions it is used in. The necessary amount of microorganism can be determined by trying out different amounts and testing the effect of the resulting product in a sensory evaluation as described herein.

The amount of microorganism may, for example, range from about 0.1% to about 1% based on the total weight of the reaction mixture. For example, the amount of microorganism used may range from about 0.1% to about 0.5% or from about 0.3% to about 0.7% based on the total weight of the reaction mixture.

The fermentation will be performed under conditions suitable for all the microorganisms involved (and all enzymes involved if occurring simultaneously with enzymatic hydrolysis). As will be apparent to the skilled person, the temperature and pH should be within a suitable range for fermentation to occur to the desired degree. The incubation length will vary accordingly, with shorter incubations when conditions are nearer to the optimum conditions. Necessary nutrients if required or beneficial for the chosen microorganisms may be present. Subjecting the incubated mixture to agitation, for example by stirring (e.g. at 50 to 500 rpm or 100 to 200 rpm) may improve the fermentation. Some microorganisms such as lactic acid bacteria may grow faster under anaerobic conditions so it may be favourable to minimize stirring. In certain embodiments, aerotolerance may be manganese-dependent.

The fermentation may, for example, be performed at a temperature less than the temperature at which the microorganisms are killed and/or reduced in numbers. The temperature may, for example, be selected to give a desired reaction rate. The fermentation may, for example, be performed at a temperature ranging from about 20° C. to about 45° C. For example, the fermentation may be performed at a temperature ranging from about 25° C. to about 40° C. or from about 30° C. to about 40° C. or from about 34° C. to about 40° C. or from about 30° C. to about 37° C. or from about 30° C. to about 35° C.

Useful temperature ranges for Lactobacilli, in particular Lactobacillus plantarum, include, for example, from about 20° C. to about 40° C., or from about 30° C. to about 40° C., or from about 35° C. to about 40° C., with an optimum of about 36° C. to about 38° C.

Useful temperature ranges for Bifidobacteria or lactic acid bacteria, in particular, L. delbrueckii ssp. bulgaricus, Streptococcus thermophiles and/or Lactobacillus acidophilus include, for example, from about 20° C. to about 40° C., or from about 30° C. to about 40° C., or from about 35° C. to about 40° C., with an optimum of about 36° C. to about 38° C. or from about 30° C. to about 35° C. or from about 30° C. to about 37° C.

Where the dietary fibre is subjected to fermentation and not enzymatic hydrolysis, the fermentation may be performed at a temperature ranging from about 30° C. to about 45° C.

The fermentation may, for example, be performed at a pH less than the temperature at which the microorganisms denature. The pH, for example, be selected to give a desired reaction rate. The fermentation may, for example, be performed at a pH ranging from about 5 to about 8, for example from about 5 to about 7 or from about 6 to about 8 or from about 6.5 to about 7.5.

The fermentation may take place for a period of time until the desired product is formed. Fermentation may, for example, take place until the fermentation medium reaches a pH of about 5.5 or lower, for example a pH of about 4.5 to about 5.5.

The fermentation may, for example, take place for a period of time ranging from about 1 day to about 10 days. For example, the fermentation may take place for a period of time ranging from about 2 days to about 9 days or from about 3 days to about 8 days or from about 4 days to about 7 days.

Where the dietary fibre is subjected to fermentation but not enzymatic hydrolysis, the fermentation may take place for a longer period of time compared to methods in which the dietary fibre is subjected to fermentation and enzymatic hydrolysis. For example, where the dietary fibre is subjected to fermentation but not enzymatic hydrolysis, the fermentation may take place for at least about 4 days. For example, where the dietary fibre is subjected to fermentation but not enzymatic hydrolysis, the fermentation may take place for a period of time ranging from about 4 days to about 10 days or from about 5 days to about 10 days or from about 6 days to about 10 days.

Where the dietary fibre is subjected to fermentation and enzymatic hydrolysis, the fermentation may take place for a shorter period of time compared to methods in which the dietary fibre is subjected to fermentation and not enzymatic hydrolysis. For example, where the dietary fibre is subjected to fermentation and enzymatic hydrolysis, the fermentation may take place for a period of time ranging from about 1 day to about 8 days or from about 2 days to about 6 days or from about 2 days to about 5 days or from about 2 days to about 4 days or from about 1 to about 2 days.

### Further Processing Steps

The product of the enzymatic hydrolysis and/or fermentation may, for example, be used directly as a flavour modifying ingredient. However, the methods may, for example, comprise one or more additional steps.

The dietary fibre that is subjected to the enzymatic hydrolysis and/or fermentation may, for example, be an aqueous slurry of dietary fibre. Thus, in certain embodiments, the method may comprise combining the dietary fibre with water prior to the enzymatic hydrolysis and/or fermentation. The aqueous slurry of dietary fibre may, for example, comprise at least about 5 wt % dietary fibre, for example at least about 10 wt % dietary fibre, for example at least about 15 wt % dietary fibre. The aqueous slurry of dietary fibre may, for example, comprise up to about 90 wt % dietary fibre or up to about 50 wt % dietary fibre or up to about 30 wt % dietary fibre.

The enzymatic hydrolysis and fermentation should be performed in a sterilized container. Thus, the container may be sterilized prior to adding the dietary fibre.

The dietary fibre (e.g. aqueous slurry of dietary fibre) may, for example, be heated prior to the enzymatic hydrolysis and/or fermentation. For example, the dietary fibre may be heated to a temperature equal to or greater than about 50° C., for example heated to a temperature in the range of 50° C. to about 55° C., or heated to a temperature equal to or greater than about 75° C., for example equal to or greater than about 100° C. or equal to or greater than about 110° C., prior to the enzymatic hydrolysis and/or fermentation. For example, the dietary fibre may be heated to a temperature equal to or less than about 140° C., for example equal to or less than about 130° C. prior to the enzymatic hydrolysis and/or fermentation. For example, the dietary fibre may be heated to a temperature of about 121° C. prior to enzymatic hydrolysis and/or fermentation. This may be to inactivate and/or kill any microbial contaminants and/or to hydrate and/or pre-heat the dietary fibre (e.g. aqueous slurry of dietary fibre) prior to enzymatic hydrolysis and/or fermentation. The dietary fibre is then maintained at a suitable temperature and/or cooled to a suitable temperature for the enzymatic hydrolysis and/or fermentation before the enzyme(s) and/or microorganism(s) are added.

The enzyme(s) and/or microorganism(s) may, for example, be deactivated prior to incorporation in a flavour composition or food product. This may, for example, take place by heating, for example to a temperature ranging from about 60° C. to about 121° C., for example about 100° C., for a period of time sufficiently long to deactivate the enzymes and/or microorganism(s). For example, any pasteurization or sterilization methods which are well-known in the art, may be used. For example, the enzymes and/or microorganisms may be deactivated by heating to about 70° C., about 90° C. or about 100° C. or higher for 30 minutes or 45 minutes or 60 minutes. When heating above about 100° C., for example about 121° C., for about 30 minutes, heating may be performed under pressure, for example about 12 to about 15 psi.

The product of the enzymatic hydrolysis and/or fermentation (the flavour modifying ingredient) may, for example, be filtered or centrifuged to remove large particles. The product of the enzymatic hydrolysis and/or fermentation (the flavour modifying ingredient) may, for example, be concentrated, for example by evaporation including boiling at, for example, up to about 100° C. The product of the enzymatic hydrolysis and/or fermentation (the flavour modifying ingredient) may, for example, be spray-dried by methods known in the art, for example using carriers such as oat fibre, soluble corn fibre, and maltodextrin and/or anti-caking agents.

Filtering may be performed by any suitable filtering method, such methods are well known in the art, for example, by passing through a felt filter bag in a filter centrifuge. The filtered culture (supernatant containing the remaining smaller solids, minus the biomass that includes larger undigested proteins) can be concentrated, for example concentrated 2× by evaporation/boiling at 100° C. The resulting concentrate's solid content can be determined using a moisture analyser and can be spray-dried, for example, onto a suitable carrier. Many carriers are well known in the art, for example, without limitation, a potato maltodextrin carrier (for example, a ratio of about 1:1 solids of the 2× concentrate to carrier may be suitable). Optionally an anti-caking agent may be added, such agents are well known. A suitable anti-caking agent is, for example, tricalciumphosphate (TPC); about 0.5% (wt/wt) based on total weight of the 2× concentrate would be a suitable amount.

The flavour modifying ingredient may, for example, be used in filtered and/or concentrated form.

The product of the enzymatic hydrolysis and/or fermentation (the flavour modifying ingredient) may, for example, be combined with one or more stabilizing agents such as propylene glycol.

### Products

The flavour modifying ingredient made by the enzymatic hydrolysis and/or the fermentation described herein may be used directly in flavour compositions and/or food compositions or may undergo further processing as described above. For example, the flavour modifying ingredient may be in filtered and/or concentrated and/or paste and/or spray-dried form. The flavour modifying ingredient may, for example, be in combination with a stabilizer such as propylene glycol, or may be in combination with one or more carriers and/or anti-caking agents used in the spray-drying process. The flavour modifying ingredient may, for example, be considered to be a natural product for food labelling and/or food regulation reasons.

The final form of the flavour modifying ingredient may be chosen according to methods well known in the art and will depend on the particular food application. For liquid foods, for example soups, the flavour modifying ingredient can be used without further processing in its liquid form. For dry applications such as crackers, the spray-dried concentrated flavour modifying ingredient can be used.

The flavour modifying ingredient may be directly added to food products, or may be provided as part of a flavour composition for flavouring or seasoning food products.

Flavour compositions contain the flavour modifying ingredient and optionally one or more food grade excipient. Suitable excipients for flavour compositions are well known in the art and include, for example, without limitation, solvents (including water, alcohol, ethanol, oils, fats, vegetable oil, and miglyol), binders, diluents, disintegranting agents, lubricants, flavouring agents, colouring agents, preservatives, antioxidants, emulsifiers, stabilisers, flavour-enhancers, sweetening agents, anti-caking agents, and the like. Examples of such carriers or diluents for flavours may be found e.g. in “Perfume and Flavour Materials of Natural Origin”, S. Arctander, Ed., Elizabeth, N.J., 1960; in “Perfume and Flavor Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

The flavour composition may contain additional flavour ingredients including flavour compounds, flavours from natural sources including botanical sources and including ingredients made by fermentation.

The flavour composition may have any suitable form, for example liquid or solid, wet or dried, or in encapsulated form bound to or coated onto carriers/particles or as a powder.

The flavour composition may, for example, comprise from about 0.02% to about 0.5% (wt/wt) based on the unconcentrated flavour modifying ingredient.

The term “food product” is used in a broad meaning to include any product placed into the oral cavity but not necessarily ingested, including, for example, food, beverages, nutraceuticals and dental care products including mouth wash.

Food products include cereal products, rice products, pasta products, ravioli, tapioca products, sago products, bakers products, biscuit products, pastry products, bread products, confectionery products, dessert products, gums, chewing gums, chocolates, ices, honey products, treacle products, yeast products, salt and spice products, savoury food products, mustard products, vinegar products, sauces (condiments), processed foods, cooked fruits and vegetable products, meat and meat products, meat analogues/substitutes/alternatives, jellies, jams, fruit sauces, egg products, dairy products (including milk), cheese products, butter and butter alternative products, milk alternative products, soy products (e.g. soy “milk”), edible oils and fat products, medicaments, beverages, juices, fruit juices, vegetable juices, food extracts, plant extracts, meat extracts, condiments, nutraceuticals, gelatins, tablets, lozenges, drops, emulsions, elixirs, syrups, and combinations thereof.

Processed foods include margarine, peanut butter, soup (clear, canned, cream, instant, UHT), gravy, canned juices, canned vegetable juice, canned tomato juice, canned fruit juice, canned juice drinks, canned vegetables, pasta sauces, frozen entrees, frozen dinners, frozen hand-held entrees, dry packaged dinners (macaroni & cheese, dry dinners-add meat, dry salad/side dish mixes, dry dinners-with meat). Soups may be in different forms including condensed wet, ready-to-serve, ramen, dry, and bouillon, processed and pre-prepared low-sodium foods.

Of particular interest are, for example, dairy products such as milk (e.g. cow's milk, goat's milk, sheep's milk, camel's milk), cream, butter, cheese, yoghurt, ice cream, and custard. The dairy products may, for example, be sweetened or unsweetened. The dairy products (e.g. milk) may, for example, be full-fat, low-fat, or non-fat.

Dairy alternative products are also of particular interest. Dairy alternative products are plant-based products that do not encompass true dairy products that have been obtained from an animal. For example, dairy alternative products include alternative “milk”, “cream”, and “yoghurt” products which may, for example, be derived from soy, almond, rice, pea, coconut, and nuts (e.g. cashew). The dairy alternative products may, for example, be sweetened or unsweetened.

Of further particular interest are, for example, beverages including beverage mixes and concentrates, including, for example, alcoholic and non-alcoholic ready to drink and dry powdered beverages, carbonated and non-carbonated beverages, e.g., sodas, fruit or vegetable juices, alcoholic and non-alcoholic beverages. The beverages may, for example, be sweetened or unsweetened.

Of further particular interest are, for example, food products traditionally high in sodium salt with a reduced sodium salt concentration, including condiments and sauces (cold, warm, instant, preserved, sate, tomato, BBQ Sauce, Ketchup, mayonnaise and analogues, bechamel), gravy, chutney, salad dressings (shelf stable, refrigerated), batter mixes, vinegar, pizza, pasta, instant noodles, french fries, croutons, salty snacks (potato chips, crisps, nuts, tortilla-tostada, pretzels, cheese snacks, corn snacks, potato-snacks, ready-to-eat popcorn, microwaveable popcorn, caramel corn, pork rinds, nuts), crackers (Saltines, ‘Ritz’ type), “sandwich-type” cracker snacks, breakfast cereals, cheeses and cheese products including cheese analogues (reduced sodium cheese, pasteurized processed cheese (food, snacks & spreads), savoury spreads, cold pack cheese products, cheese sauce products), meats, aspic, cured meats (ham, bacon), luncheon/breakfast meats (hotdogs, cold cuts, sausage), soya-based products, tomato products, potato products, dry spice or seasoning compositions, liquid spice or seasoning compositions including pesto, marinades, and soup-type/meal-alternative beverages, and vegetable juices including tomato juice, carrot juice, mixed vegetable juices and other vegetable juices.

The food product may, for example, comprise from about 0.001% to about 0.5 (wt/wt) based on the unconcentrated flavour modifying ingredient, for example from about 0.001% to about 0.02% (wt/wt) based on the unconcentrated flavour modifying ingredient.

If the flavour modifying ingredient is added as an unconcentrated liquid, about 0.005 to about 0.5% (wt/wt) is usually enough, for example, without limitation, in soups and topical food applications such as chips, crisps and snacks.

Depending on the food product more may be needed. For most topical applications, about 0.1% to about 0.5% (wt/wt) is sufficient. When using a concentrate (for example by distillation) or a spray-dried salt enhancing ingredient, the concentrations indicated need to be adjusted with an appropriate factor to take into account of the concentration change in the salt enhancing ingredient.

Depending on the food product, for food products that contain about 10 to 100%, for example 25 to 50%, less sodium than a comparable food product (for example “reduced sodium” products with 25% reduction, or “light in sodium” products with a 50% reduction), the flavour modifying ingredient may be employed as follows: a useful concentration for most food applications may be, for example, about 0.001% to about 0.015% (wt/wt) based on the unconcentrated flavour modifying ingredient. Alternatively, for example, 25 to 300 ppm or 0.002% to 0.03% (wt/wt) based on a spray-dried 2× concentrate may be used.

The flavour modifying ingredient may be used in unconcentrated or concentrated form or the concentrate may be formulated into a paste or powder by methods known in the art. In this case the amount to be used has to be adjusted accordingly. Flavour compositions such as spices are often more concentrated, for example a 10× concentrate, and the concentration will be adjusted higher accordingly (250 ppm to 3000 ppm).

The NaCl concentration in common food products with a regular NaCl concentration varies with most products ranging from about 0.5% to about 5% (wt/wt) NaCl. Seasoning or products used as seasoning, such as croutons, sauces or salad dressings that are employed in a small amount (to be applied to, for example, salad or noodles), have a concentration of for example from about 2% to about 5% (wt/wt) NaCl. Soups usually contain about 0.6% to about 1.25% (wt/wt) NaCl. Salty crackers and meat products (e.g. salami, ham, and bacon) usually contain about 2% to about 4% (wt/wt) NaCl. Cereals usually contain about 0.6 to 3% (wt/wt) NaCl. Products that need to be reconstituted (dry soups) usually range in the concentration ranges indicated after reconstitution.

For low sodium products containing even less NaCl than products with reduced sodium content (e.g. 353 mg per serving), the amount of the salt enhancing ingredient may have to be increased.

For food products with added KCl, depending on the food product and said ingredients, the concentration of KCl may be from about 0.1% or about 0.2%, up to about 1%, up to about 1.5%, up to about 2% (wt/wt), or higher, depending on how much the sodium concentration is reduced. A KCl concentration of about 0.25% to about 1.5% (wt/wt), for example about 0.5% to about 1.5% (wt/wt) KCl will be useful for most low sodium products. A range to which the NaCl concentration may usefully be reduced for most applications is, for example, about 0.25% (wt/wt) to about 2.5% (wt/wt), or from about 0.125% to about 1.25% (wt/wt). The amount of the flavour modifying ingredient to be added to the food product as an ingredient will depend on the concentration of KCl used, and the specific food product including the particular base and flavour. A useful concentration for most food applications may be, for example, about 0.001% to about 0.015% (wt/wt) based on the unconcentrated flavour modifying ingredient. Alternatively, for example, 25 to 300 ppm or 0.002% to 0.03% (wt/wt) based on a spray-dried 2× concentrate may be used.

The flavour modifying ingredient may be used in un-concentrated form or the concentrate may be formulated into a paste or powder or spray-dried salt enhancing ingredient by methods known in the art. In this case, the amount to be used has to be adjusted accordingly.

The appropriate concentration of the flavour modifying ingredient can be easily tested by an organoleptic titration. This technique is well known in the field of sensory analysis.

The flavour compositions and food products may, for example, comprise one or more sweeteners. Examples of sweeteners that may be used in the sweetened compositions are disclosed, for example, in WO 2016/038617, the contents of which are incorporated herein by reference.

The one or more sweeteners may, for example, be selected from sucrose, fructose, glucose, xylose, arabinose, rhamnose, tagatose, allulose, trehalose, isomaltulose, steviol glycosides (e.g. rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, dulcoside B, rubusoside, naringin dihydrochalcone, stevioside), mogrosides (e.g. grosvenorine II, grosvenorine I, 11-O-mogroside II (I), 11-O-mogroside II (II), 11-O-mogroside II (III), mogroside II (I), mogroside II (II), mogroside II (III), 11-dehydroxy-mogroside III, 11-O-mogroside III, mogroside III (I), mogroside III (II), mogroside IIIe, mogroside IIIx, mogroside IV (I) (siamenoside), mogroside IV (II), mogroside IV (III), mogroside IV (IV), deoxymogroside V (I), deoxymogroside V (II), 11-O-mogroside V (I), mogroside V isomer, mogroside V, iso-mogroside V, 7-O-mogroside V, 11-O-mogroside VI, mogroside VI (I), mogroside VI (II), mogroside VI (III) (neomogroside) and mogroside VI (IV)), stevia, trilobatin, rebusoside, aspartame, advantame, agarve syrup, acesulfame potassium (AceK), high fructose corn syrup, neotame, saccharin, sucralose, high fructose corn syrup, starch syrup, Luo Han Guo extract, neohespiridin, dihydrochalcone, naringin, sugar alcohols (e.g. sorbitol, xylitol, inositol, mannitol, erythritol), cellobiose, psicose, and cyclamate.

### Uses

The flavour modifying ingredient obtained by and/or obtainable by the methods described herein may, for example, be added to food products (e.g. as part of a flavour composition) to modify the flavour or mouthfeel of the food product.

The flavour modifying ingredient obtained by and/or obtainable by the methods described herein may, for example, be used to improve the mouthfeel of a food product and/or to mask off-notes of a food product and/or to improve the sweetness of a food product and/or to enhance the saltiness of a food product and/or to act as a prebiotic in a food product.

Thus, there is also provided herein a method of providing a food product having improved mouthfeel and/or reduced off-notes and/or improved sweetness and/or enhanced saltiness and/or use as a prebiotic, the method comprising admixing the flavour modifying ingredient obtained by and/or obtainable by the methods described herein with the food product.

In general terms, “mouthfeel” refers to the complexity of perceptions experienced in the mouth as influenced by the aroma, taste, and texture qualities of food and beverage products. From a technical perspective, however, mouthfeel sensations are specifically associated with physical (e.g. tactile, temperature) and/or chemical (e.g. pain) characteristics perceived in the mouth via the trigeminal nerve. Accordingly, they are a consequence of oral-tactile stimulations and involve mechanical, pain and temperature receptors located in the oral mucosa, lips, tongue, cheeks, palate and throat.

Mouthfeel perceptions include, for example, one or more of texture—astringent, burning, cold, tingling, thick, biting, fatty, oily, slimy, foamy, melting, sandy, chalky, watery, acidic, lingering, metallic, body, body sweet, carbonation, cooling, warming, hot, juicy, mouth drying, numbing, pungent, salivating, spongy, sticky, fullness, cohesiveness, density, fracturability, graininess, grittiness, gumminess, hardness, heaviness, moisture absorption, moisture release, mouthwatering, mouthcoating, roughness, slipperiness, smoothness, uniformity, uniformity of bite, uniformity of chew, viscosity, fast-diffusion, full body, salivation and retention.

As stated previously, the perceived mouthfeel of a food or beverage can be broadly influenced by the presence of aroma and taste attributes in addition to textural properties. Thus a number of other attributes may affect the experienced overall mouthfeel sensation of a product including, for example, one or more of taste or aroma—for example sweet, salty, umami, sour, bitter, creamy sour, acidic, acidic dairy, green onion, toasted onion and parsley.

By “improvement of mouthfeel” it is meant that any one or more of desired mouthfeel perceptions is/are enhanced and/or that any one or more undesirable mouthfeel perceptions is/are reduced. In particular, one or more of the following perceptions may be enhanced by the product and methods described herein: creamy sour, acidic dairy, sweet, salty, umami.

By “masking of off-notes” it is meant that the intensity and/or length of perception of undesirable attributes in a food product is reduced, as analysed by trained panellists when comparing food comprising an ingredient with off-note masking to food without an added off-note masking ingredient.

By “improvement in sweetness” it is meant the effect of the flavour modifying ingredient on the sweetness characteristics of a food which are found to be more favourable as analysed by trained panellists when comparing food comprising an ingredient with sweetness improving effect to food without an added sweetness improving ingredient.

The improvement in sweetness may, for example, provide sweetness characteristics that are more similar to the sweetness characteristics of sucrose.

The sweetness characteristics may refer to the flavour profile (taste profile), which refers to the intensity of the flavour and perceptual attributes of a given compound. Exemplary flavour attributes of sweetness are sweetness intensity, bitterness, black liquorice etc.

The sweetness characteristics may refer to the temporal profile, which refers to the changes in perception of sweetness over time. Every sweetener exhibits a characteristic appearance time (AT) and extinction time (ET). Most high-potency sweeteners, in contrast to carbohydrate sweeteners, display prolonged ET (lingering). Generally, the detected sucrose equivalence spikes to a maximal response level, then tapers off over time. The longer the taper, the greater the detected sweetness linger of a compound.

The improvement in sweetness may, for example, be particularly obtained when the flavour modifying ingredient is used in sweetened food products. The improvement of sweetness may, for example, be particularly obtained in dairy products or beverages, for example sweetened dairy products or beverages.

In certain embodiments, the flavour modifying ingredient may be used to weaken the lingering sweet taste of the food product (e.g. sweetened food product). In other words, the flavour modifying ingredient may be used to decrease the extinction time (ET) of the food product (e.g. sweetened food product). This relates to the undesirable lingering of the sweetness taste in the mouth after the food product is initially ingested or expectorated. The lingering sweet taste may, for example, refer to the length of time that the sweetness taste remains after it is initially detected, how rapidly the intensity of the sweetness taste decreases or fades after it is initially detected and the intensity of the sweetness taste after it is initially detected. The flavour modifying ingredient may, for example, decrease the length of time that the sweetness taste remains after it is initially detected and/or increase the speed at which the sweetness taste decreases after it is initially detected and/or decrease the intensity of the sweetness taste after it is initially detected.

In certain embodiments, the flavour modifying ingredient may be used to weaken the bitter taste and/or astringent taste and/or metallic taste and/or liquorice taste of the food product (e.g. sweetened food product).

In certain embodiments, the flavour modifying ingredient may be used to strengthen the sweetness impact of the food product (e.g. sweetened food product). The sweetness impact relates to the length of time it takes before the sweetness is initially detected and the intensity at which the sweetness is initially detected. The flavour modifying ingredient may, for example, decrease the amount of time before the sweetness is initially detected and/or increase the intensity at which the sweetness is initially detected.

The degree of sweetness and other sweetness characteristics described herein may be evaluated by a tasting panel of trained experts, for example as described in the examples below.

By “salt enhancing” it is meant the effect of the flavour modifying ingredient on the salt taste of a food which is found more pronounced (stronger, enhanced) in its taste intensity and and/or longer in its duration as analysed by trained panellists sensitive to salty taste when comparing food comprising an ingredient with a salt enhancing effect to food without an added salt enhancing ingredient.

By “prebiotic” it is meant the effect of the flavour modifying ingredient to improve the effect of gut flora, for example by increasing the activity of the gut flora and/or by increasing the population of the gut flora.

### EXAMPLES

### Example 1—Fermented Oat Fibres

Flavour modifying ingredients were made by fermenting oat fibres by the following process.

831 g of water was added to a clean, sanitized tank. 166 g of oat fibre (AvenOLait™ oat fibre obtained commercially from Axiom Foods Inc.) was added to the water. The mixture was heated to 121° C. within 1 hour and with continuous mixing. The temperature of the mixture was held at 121° C. for 30 minutes. The mixture was then cooled to 37° C. before 3 g of seed ferment was added. The mixture was incubated at 37° C. with slow agitation for four days. The mixture was then pasteurized at 100° C. for 45 minutes. The product was stored at 4° C.

The seed ferment used was either rice coated with Aspergillus oryzae obtained from Rhapsody Natural Foods, Cabot VT 05647 (Flavour Modifying Ingredient (FMI)-A) or Lactobacillus plantarum ATCC 14917.

Organoleptic evaluation was undertaken using each flavour modifying ingredient in various food products at a concentration of about 0.1% (pea yoghurt, non-fat yoghurt, soy yoghurt, and 2% fat milk).

In the potato chips, the flavour modifying ingredient was used in the sour cream and onion base seasoning at a concentration of 0.1% and the sour cream and onion base seasoning was added to the potato chips at a concentration of 7%.

The various food products were as follows:

Pea Yoghurt (Ripple, Original—dairy alternative product)
Non-Fat Yoghurt (obtained from Danone—non-sweetened, non-fat dairy product)
Soy Yoghurt (Silk, plain—dairy alternative product)
2% Fat Milk (obtained from Kroger—non-sweetened, low fat dairy product)
Potato chips (Mike Sells Original Unsalted Chips)+7% sour cream and onion base flavour

Organoleptic evaluation of the pea yoghurt, non-fat yoghurt, soy yoghurt, and 2% fat milk was carried out by flavorists (descriptive analysis).

Organoleptic evaluation of the sour cream and onion chips was carried out by a paired comparison strategy, whereby potato chips with the sour cream and onion base flavour with FMI-A were compared to potato chips with the sour cream and onion base flavour without FMI-A. Eleven panellists performed a pre-evaluation review and training with the sour cream and onion potato chips. For the organoleptic evaluation, samples were presented to the panellists in pairs as blinded samples in a randomized, fully balanced order. For each pair of products, the panellist was instructed to select the sample that was greater for each attribute (creamy sour, green onion, toasted onion, parsley, acidic dairy, sweet, salty, umami). Four repetitions of each paired evaluation were performed.

The definition of the attributes tested for the organoleptic evaluation of the sour cream and onion chips was as follows.

Creamy sour: Sour dairy aroma associated with sour cream, butter, and yoghurt
Green onion: Herbal, green, alliaceous aroma associated with green onions
Toasted onion: Sweet, brown, toasted, and alliaceous aroma associated with onion powder
Parsley: Green, leafy, and woody aroma associated with fresh parsley leaves
Acidic dairy: Basic taste on the tongue associated with lactic acid in solution, similar to fermented cow's milk
Sweet: Basic taste sensation associated with sugars and high potency sweeteners in solution
Salty: Basic taste sensation associated with table salt (NaCl) diluted in water
Umami: Basic taste sensation associated with MSG, characterized by fullness of flavour in the mouth, often found in bouillons, soy sauce, and mushrooms

The results are provided below.

### Pea Yoghurt

FMI-A Taste Evaluation: Cleaned pea note, masks astringency, good cultured note profile, cleaner, sour (preferred compared to FMI-B).

FMI-B Taste Evaluation: Sweeter, less astringent, masks pea note, creamy, acid is masked, sweeter note comes through, less gritty.

### Non-Fat Yoghurt

FMI-A Taste Evaluation: Very acidic, more sour, best cultured note, clean, more cultured.

FMI-B Taste Evaluation: Makes more balanced yogurt profile, cleaner acid note, more cultured, more dairy note, very acidic sharp, balanced, clean finish, more cultured, more sour note (Preferred compared to FMI-A).

### Soy Yoghurt

FMI-B Taste Evaluation: Good yogurt profile, less astringent, masks beany, creamy, sweet, nice upfront, acidic middle end, slight cultured, very smooth, balanced acidity. 2% Fat Milk

FMI-B Taste Evaluation: Fatty full, almost like full fat milk, yogurt like, creamy, cultured at the end, clean profile.

### Sour Cream & Onion Chips

FMI-A Taste Evaluation: Creamy note enhancements, saltier than base alone (p<0.05), less vegetative notes (parsley) than base alone (p<0.05), increased perception of umami, acidic dairy, and toasted onion than base alone (p<0.1).

It was surprisingly found that the flavour modifying ingredients eliminated the unpleasant beany taste of the dairy alternative products (pea yoghurt and soy yoghurt).

In addition, it was surprisingly found that the flavour modifying ingredients provided a “fullness” sensation to the low-fat or non-fat dairy products (non-fat yoghurt and 2% fat milk) that gives the impression of the corresponding full-fat dairy product.

It was further surprisingly found that the flavour-modifying ingredient FMI-A provided a salty taste in a savoury product (sour cream and onion chips).

### Example 2—Enzymatic Hydrolysis of Grape Fibre

A Flavour modifying ingredient was made by subjecting grape fibre to enzymatic hydrolysis.

The flavour modifying ingredient (FMI-C) was made by mixing 70 g of Concord Grape Fibre (obtained from FruitSmart) with 623.35 g of water in a clean, sanitized tank. The following enzymes were then added to the mixture: 3.5 g Celluclast® (from Novozyme), 1.4 g Viscozyme® (from Novozyme), 0.7 g Flavorzyme® (from Novozyme), 0.35 g Umamizyme® (from Amano Enzymes), and 0.7 g Ceramix® (from Novozyme). The mixture was incubated at 50° C. for 24 hours with continuous agitation.

FMI-C was then filter-centrifuged through a felt filter bag at 1000 rpm for 10 minutes to remove large solids. The filtrate (532 g) was heated at 100° C. for 1 hour to inactivate the enzymes. FMI-C was then stabilized by mixing with 228 g of propylene glycol and stored at 4° C.

Organoleptic evaluation was undertaken using FMI-C in various Reb A-, sucralose- and sugar-sweetened beverage bases at a concentration of 0.07% and 0.09%.

The Reb A-, sucralose- and sugar-sweetened bases were as follows:

Hybrid RebA-sugar base (a still neutral beverage sweetened with RebA-sugar for 30% sugar reduction—7.3% sugar+0.1% citric acid)

Reduced sugar base (5% sucrose+0.03% citric acid in water; benchmark: 5.5% sucrose+0.03% citric acid in water)

Hybrid Sucrose-Glucose-Fructose-RebA base (0.9% sucrose, 0.45% glucose, 0.45% fructose and 180 ppm RebA+0.05% citric acid in water; benchmark: 1.4% sucrose, 0.7% glucose, 0.7% fructose and 120 ppm RebA+0.05% citric acid in water)

Hybrid Sucralose-AceK base (70 ppm sucralose and 21 ppm AceK+0.05% citric acid in water; benchmark: 35 ppm sucralose, 21 ppm AceK and 2.5% sucrose+0.05% citric acid in water)

Organoleptic evaluation was carried out by groups of 2-4 flavorists, comparing the samples containing FMI-C to their corresponding bases and benchmarks (paired comparison).

It was found that when FMI-C was added at a concentration of 0.09%, there was improved upfront sweetness and deceased linger of the hybrid RebA-sugar sweetened base.

When FMI-C was added at a concentration of 0.07%, there was increased sweetness of the Reduced sugar base containing 5% sucrose by about ½ brix.

### Example 3—Enzymatic Hydrolysis and Fermentation of Cranberry Fibre

A Flavour modifying ingredient was made by subjecting cranberry fibre to enzymatic hydrolysis followed by fermentation.

The flavour modifying ingredient (FMI-D) was made by mixing 105 g of the Cranberry fibre (obtained from FruitSmart) with 589.4 g of water in a clean, sanitized tank. The following enzymes were then added to the mixture: 3.5 g Celluclast® (from Novozyme), 1.4 g Viscozyme® (from Novozyme), 0.7 g Flavorzyme® (from Novozyme), 0.35 g Umamizyme® (from Amano Enzymes), and 0.7 g Ceramix™ (from Novozyme) and the mixture was then incubated at 50° C. for 24 hours with continuous agitation. The mixture was then cooled to 37° C. and 3.5 g of Aspergillus oryzae (Koji) culture was added (obtained from Rhapsody Natural Foods). The mixture was incubated at 37° C. for 96 hours with agitation and open ports.

The slurry was then diluted with water to bring the solids content from 15% to 10% and filter-centrifuged through a felt filter bag at 1000 rpm for 10 minutes to remove large solids. The filtrate (511 g) was then heated at 100° C. for 1 hour to inactivate the enzymes. FMI-D was then stabilized by mixing with 219 g of propylene glycol and stored at 4° C.

Organoleptic evaluation was undertaken using FMI-D at a concentration of 0.05% in a zero-calorie base containing 180 ppm Reb A and 0.05% citric acid (descriptive analysis).

Organoleptic evaluation was carried out by 6 panelists (flavorists and scientists).

It was found that addition of FMI-D to the zero-calorie base resulted in reduced lingering, masked bitterness and metallic taste, and a more sugary mouthfeel.

Organoleptic evaluation was also undertaken using 0.075% FMI-D in plain pea yoghurt obtained from Ripple foods sweetened with 180 ppm Reb A.

FMI-D provided sweetness, creaminess, and a more clean taste profile compared to the blind blank base.

### Example 4—Enzymatic Hydrolysis of Grape Fibre

A Flavour modifying ingredient was made by subjecting grape fibre to enzymatic hydrolysis.

The flavour modifying ingredient (FMI-E) was made by mixing 140 g of the Concord Grape fibre (obtained from FruitSmart) with 547.9 g of water in a clean, sanitized tank. The following enzymes were then added to the mixture: 5.25 g Celluclast® (from Novozyme), 2.1 g Viscozyme® (from Novozyme), 1.05 g Flavorzyme® (from Novozyme), 0.525 g Umamizyme® (from Amano Enzymes), 1.05 g Ceramix™ (from Novozyme), 1.4 g Hemicellulase (from Amano Enzymes), and 0.7 g Mannanase (from Amano Enzymes). The mixture was incubated at 50° C. for 24 hours with continuous agitation.

The slurry was then filter-centrifuged through a felt filter bag at 1000 rpm for 10 minutes to remove the large solids. The filtrate (429 g) was heated at 100° C. for 1 hour to inactivate the enzymes and the ingredient was stabilized by mixing with 184 g Propylene glycol and stored at 4° C.

Organoleptic evaluation was undertaken using FMI-E in various Reb A-, sucralose- and sugar-sweetened beverage bases at concentrations between 0.02% and 0.09%

The Reb A-, sucralose- and sugar-sweetened bases were as follows with the same composition as the bases used in Example 2:

Reduced sugar base (5% sucrose+0.03% citric acid)

Hybrid Sucrose-Glucose-Fructose-RebA base

Hybrid Sucralose-AceK base

Organoleptic evaluations were carried out by 6 flavorists.

It was found that at 0.09% concentration, FMI-E increased the sweetness of the reduced-sugar base (containing 5% sucrose) by more than ½ brix. At 0.045% concentration, the sweetness of the reduced-sugar base (containing 5% sucrose) increased by about ½ brix. At 0.03% concentration, FMI-E added body in the middle of the sweetness and modulated (reduced) metallic linger of sucralose in the hybrid sucralose-AceK-sugar base.

### Example 5—Enzymatic Hydrolysis and Fermentation of Oat Fibre

A Flavour modifying ingredient or probiotic drink was made by subjecting oat fibre to enzymatic hydrolysis and fermentation.

The flavour modifying ingredient or probiotic drink was made by mixing Oat flour (code name P12 or BG28 from Naturex) or Oat kernels (from Grain Millers Inc US) with water to form a slurry with 20 to 30% solids. The aqueous slurry was heated to a temperature in the range of 50° C. to about 55° C. prior to the enzymatic hydrolysis. Alpha-Amylase enzyme (Kleistase® SD-80, from Amano Enzyme at a concentration of 1 to 1.5%) was then added and the mixture was incubated at 50-55° C. for 2 hours to break down the amylose and amylopectin to maltose and various dextrins. Glucoamylase (Gluczyme®NLP from Amano Enzyme at a concentration of 0.5 to 1.5% for another 1 to 2 hrs at 50° C. to 55° C.) was then added for further breakdown to release glucose. Protease/aminopeptidase enzymes (Protein Glutaminase®500 from Amano) were also added to hydrolyze the proteins at 50° C. to 55° C. for 1 to 2 hours. The mixture was pasteurized at 100° C. for 45 minutes to inactivate all enzymes. Then it was fermented for 24 to 48 hours at 30-35° C. with lactic acid bacteria (such as L. delbrueckii ssp. bulgaricus, Streptococcus thermophilus, Lactobacillus acidophilus) and/or a Bifidobacterium at a concentration of 0.3 to 0.7%. The cultures were obtained from a commercial supplier (eg, Vivolac, US) as a frozen concentrate. The resulting flavour modifying ingredient was then either kept refrigerated or further processed by spray-drying.

Organoleptic evaluation was undertaken by adding the flavour modifying ingredient at a concentration of 0.05% to GoodBelly's® dairy-free probiotic shots. Six panellists carried out the organoleptic evaluation. All panellists found that the flavour modifying ingredient provided good body and improved mouthfeel as well as off-note masking and some sweetness improvement. The results are provided in the Table below.

Evaluation of enzymatically hydrolyzed and fermented Oat fibers at a
concentration of 0.05% in GoodBelly ® dairy-free probiotic shots

| Sample code # | Fiber material | Comments |
|---|---|---|
| 229-9220-00 | Oat kernel | Good mouthfeel, slight sweetness. |
| 229-9228-00 | Oat flour BG28 | Good mouthfeel, viscous texture, |
| | | good masking, more dairy like, |
| | | creamy, smooth |
| 229-9230-00 | Oat flour P12 | Good mouthfeel, less off note, |
| | | slight sweet, dairy like, fullness, |
| | | cleaner |

The foregoing broadly describes certain embodiments of the present invention without limitation. Variations and modifications as will be readily apparent to those skilled in the art are intended to be within the scope of the present invention as defined in and by the appended claims.

1. A method for making a flavour modifying ingredient, the method comprising subjecting a dietary fibre to enzymatic hydrolysis or fermentation, or enzymatic hydrolysis and fermentation, wherein the fermentation uses one or more than one lactic acid bacteria selected from the group consisting of Lactobacillus plantarum, Lactobacillus casei, Lactobacillus brevis, Lactobacillus helveticus, L. delbrueckii ssp. bulgaricus, Streptococcus thermophiles, Lactobacillus acidophilus and/or Bifidobacterium.

2. The method of claim 1, wherein the dietary fibre is isolated dietary fibre.

3. The method of claim 1, wherein the dietary fibre is an aqueous slurry of dietary fibre.

4. The method of claim 1, wherein the dietary fibre is a cereal fibre, a vegetable fibre, or a fruit fibre.

5. The method of claim 1, wherein the enzymatic hydrolysis uses one or more enzymes selected from carbohydrases and proteolytic enzymes.

6. The method of claim 1, wherein the enzymatic hydrolysis uses at least one or more enzymes selected from cellulases, pectinases, and other carbohydrases.

7. (canceled)

8. The method of claim 1, wherein the enzymatic hydrolysis is performed at a temperature ranging from about 25° C. to about 60° C.

9. The method of claim 1, wherein the enzymatic hydrolysis takes place for a period of time ranging from about 1 hour to about 48 hours.

10. The method of claim 1, wherein the fermentation is performed at a temperature ranging from about 20° C. to about 45° C.

11. The method of claim 1, wherein the fermentation takes place for a period of time ranging from about 1 day to about 10 days.

12. (canceled)

13. The method of claim 1, wherein the enzymatic hydrolysis occurs before and/or simultaneously with the fermentation.

14. (canceled)

15. The method of claim 1, wherein the method comprises subjecting a dietary fibre to enzymatic hydrolysis and does not comprise subjecting the dietary fibre to fermentation.

16. (canceled)

17. (canceled)

18. (canceled)

19. The method of claim 1, wherein the method comprises subjecting a dietary fibre to fermentation and does not comprise subjecting the dietary fibre to enzymatic hydrolysis.

20. (canceled)

21. (canceled)

22. The method of claim 19, wherein the fermentation takes place for a period of time ranging from about 4 days to about 10 days.

23. The method of claim 1, wherein the method comprises heating the dietary fibre to a temperature equal to or greater than about 75° C. prior to the enzymatic hydrolysis and fermentation.

24. (canceled)

25. The method of claim 1, wherein the method further comprises combining the flavour modifying ingredient with propylene glycol.

26. The method of claim 1, wherein the method further comprises spray-drying the flavour modifying ingredient.

27. A flavour modifying ingredient obtainable by and/or obtained by the method of claim 1.

28. (canceled)

29. (canceled)

30. (canceled)

31. (canceled)

32. (canceled)

33. A food product comprising the flavour modifying ingredient of claim 27.

34. (canceled)

35. The food product of claim 33, wherein the food product is a dairy product or a dairy alternative product or a beverage or a savoury food.

36. (canceled)

37. (canceled)

38. (canceled)

39. A method of providing a food product having an improved mouthfeel, the method comprising admixing the flavour modifying ingredient of claim 27 to the food product.

40. (canceled)

41. A method of providing a food product having reduced off-notes, the method comprising admixing the flavour modifying ingredient of claim 27 to the food product.

42. (canceled)

43. A method of providing a food product having improved sweetness, the method comprising admixing the flavour modifying ingredient of claim 27 to the food product.

44. (canceled)

45. (canceled)

46. (canceled)

47. A method of providing a prebiotic food product, the method comprising admixing the flavour modifying ingredient of claim 27 to the food product.

48. (canceled)

49. The method of claim 4, wherein the cereal fibre is oat fibre; the vegetable fibre is pea fibre; and the fruit fibre is selected from the group consisting of citrus fruit fibre, apple fibre, blueberry fibre, cranberry fibre, grape fibre, and combinations thereof.
